(19) United States
(12) Patent Application Publication
(10) Pub. No.: US 2009/0029050 A1
(43) Pub. Date: Jan. 29, 2009

(54) ARYLIDENEPYRAZOLONE DYE, HEAT-SENSITIVE TRANSFER RECORDING INK SHEET AND HEAT-SENSITIVE TRANSFER RECORDING METHOD

(75) Inventors: Kimihiko SATO, Ashigarakami-gun (JP); Yoshihiko Fujie, Minami-ashigara-shi (JP); Shinichi Ichikawa, Minami-ashigara-shi (JP); Takashi Hoshimiya, Minami-ashigara-shi (JP); Naotsugu Muro, Minami-ashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/180,889

(22) Filed: Jul. 28, 2008

(30) Foreign Application Priority Data
Jul. 27, 2007 (JP) 2007-196271

(51) Int. Cl. B41M 5/40; B05D 5/00; C07D 231/08

(52) U.S. Cl. 427/256; 428/32.6; 548/369.4

(57) ABSTRACT

A heat-sensitive transfer recording ink sheet, containing a base material sheet and a dye-providing layer formed, wherein at least one kind of yellow dye contained in the dye-providing layer is an arylidenepyrazolone dye represented by formula (1):

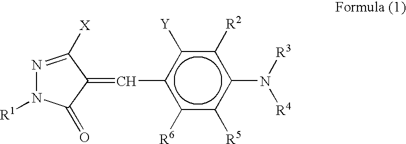

wherein X represents a substituted or unsubstituted alkoxy group having 1 to 6 carbon atoms; Y represents a halogen atom; R¹ represents a substituted or unsubstituted aryl group having 6 to 10 carbon atoms; R², R⁵ and R⁶ each represent a hydrogen atom, an unsubstituted alkyl group having 1 to 4 carbon atoms, an unsubstituted alkoxy group having 1 to 4 carbon atoms, or a halogen atom; and R³ and R⁴ each represent a substituted or unsubstituted alkyl group having 1 to 5 carbon atoms, or an allyl group.

### FIELD OF THE INVENTION

The present invention relates to a novel specific arylidenepyrazolone dye compound, a heat-sensitive transfer recording ink sheet and a cartridge containing the dye compound, and a heat-sensitive transfer recording method using them.

### BACKGROUND OF THE INVENTION

In recent years, in particular, materials for forming a color image have been mainly used as an image recording material. Specifically, recording materials of inkjet system, recording materials of heat-sensitive transfer system, recording materials of electrophotographic system, silver halide photosensitive materials of transfer system, printing inks, recording pens, and the like, have been used extensively. Color filters are used in image devices, such as image pick up device like CCD (Charge Coupled Device), and in displays, such as LCD (Liquid Crystal Display) and PDP (Plasma Display Panel), to record and reproduce color images.

In these color image recording materials and color filters, colorants (dyes or pigments) of three primary colors are used based on a so-called additive color mixing system or subtractive color mixing system, to reproduce or record full-color images. A colorant, which has absorption characteristics that enable the acquisition of a preferred color reproduction range and has fastness under various conditions for use, has not been available yet, and its improvement is strongly desired.

As the heat-sensitive transfer recording method, there are a process including the steps of heating a heat-sensitive transfer material having a base material sheet (hereinafter, also referred to as “support” or “base film”) and a hot-melt ink layer formed thereon with a thermal head, and recording the melted ink to an image-receiving material; and a process including the steps of heating a heat-sensitive transfer material having a support and a dye-providing layer formed thereon that contains a heat transfer dye with a thermal head, and thermal-diffusionally transferring the dye onto an image-receiving material. The latter heat-sensitive transfer process is able to change a transfer amount of the dye by altering energy applied to a thermal head, so that a gradation recording is easily achieved. Consequently, such the process is especially advantageous to a high quality full color recording. However, because the heat transfer dye usable in the process is limited in various points, only a considerably few dyes satisfy all the performances required for the process.

Performance requirements for the dyes include bearing spectral characteristics desirable for color reproduction, causing sublimation and/or transfer by a thermal recording head, having a high molecular extinction coefficient, being fast to light and heat, resisting attack by various chemicals, having easiness of synthesis, ensuring easy production of heat-sensitive transfer recording materials, and being safe. However, the special dyes hitherto proposed as those having spectral characteristics desirable for color reproduction and fastness to light and heat (see, for example, JP-A-63-189289 (“JP-A” means unexamined published Japanese patent application) and JP-A-60-53564) are not on satisfactory levels, and further improvements are desired strongly.

Dyes having a particular arylidenepyrazolone skeleton for heat-sensitive transfer recording are proposed (see, for example, JP-A-2-3450). However, those dyes are not on satisfactory levels in terms of the required performance characteristics, so further studies are needed.

### SUMMARY OF THE INVENTION

The present invention resides in a heat-sensitive transfer recording ink sheet, containing a base material sheet and a dye-providing layer formed on one side of the base material sheet, wherein at least one kind of yellow dye contained in the dye-providing layer is an arylidenepyrazolone dye represented by formula (1):

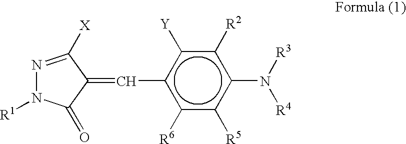

wherein X represents a substituted or unsubstituted alkoxy group having 1 to 6 carbon atoms; Y represents a halogen atom; R¹ represents a substituted or unsubstituted aryl group having 6 to 10 carbon atoms; R², R⁵ and R⁶ each independently represent a hydrogen atom, an unsubstituted alkyl group having 1 to 4 carbon atoms, an unsubstituted alkoxy group having 1 to 4 carbon atoms, or a halogen atom; R³ and R⁴ each independently represent a substituted or unsubstituted alkyl group having 1 to 5 carbon atoms, or an allyl group; and R³ and R⁴ and/or R⁵ and R⁶ may combine with each other to form a 5- or 6-membered ring.

Further, the present invention resides in an ink sheet cartridge, which is loaded with the above-described heat-sensitive transfer recording ink sheet as described above.

Further, the present invention resides in a heat-sensitive transfer recording method, wherein the above-described heat-sensitive transfer recording ink sheet is used with an image-receiving material having an ink-receiving layer containing a polymer on a support, to form an image.

Furthermore, the present invention resides in an arylidenepyrazolone dye compound represented by formula (2):

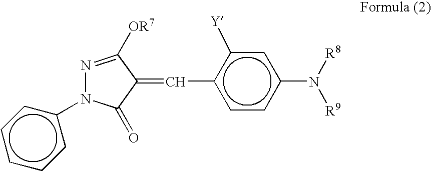

wherein Y′ represents a fluorine atom, a chlorine atom or a bromine atom; R⁷ represents a methyl group, an ethyl group, an n-propyl group or an isopropyl group; and R⁸ and R⁹ each independently represent a methyl group, an ethyl group, an n-propyl group, an isopropyl group or an allyl group.

Other and further features and advantages of the invention will appear more fully from the following description.

### DETAILED DESCRIPTION OF THE INVENTION

According to the present invention, there is provided the following means:
(1) A heat-sensitive transfer recording ink sheet comprising a base material sheet and a dye-providing layer formed on one side of the base material sheet, wherein at least one kind of yellow dye contained in the dye-providing layer is an arylidenepyrazolone dye represented by formula (1):

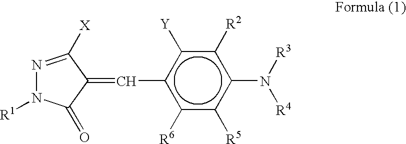

wherein X represents a substituted or unsubstituted alkoxy group having 1 to 6 carbon atoms; Y represents a halogen atom; R¹ represents a substituted or unsubstituted aryl group having 6 to 10 carbon atoms; R², R⁵ and R⁶ each independently represent a hydrogen atom, an unsubstituted alkyl group having 1 to 4 carbon atoms, an unsubstituted alkoxy group having 1 to 4 carbon atoms, or a halogen atom; R³ and R⁴ each independently represent a substituted or unsubstituted alkyl group having 1 to 5 carbon atoms, or an allyl group; and R³ and R⁴ and/or R⁵ and R⁶ may combine with each other to form a 5- or 6-membered ring.
(2) The heat-sensitive transfer recording ink sheet as described in the above item (1), wherein X is an unsubstituted alkoxy group having 1 to 3 carbon atoms, Y is a fluorine atom, a chlorine atom or a bromine atom, R¹ is a substituted or unsubstituted phenyl group, R², R⁵ and R⁶ each are a hydrogen atom, and R³ and R⁴ each are a substituted or unsubstituted alkyl group having 1 to 3 carbon atoms, or an allyl group.
(3) The heat-sensitive transfer recording ink sheet as described in the above item (1), wherein the arylidenepyrazolone dye represented by formula (1) is an arylidenepyrazolone dye represented by formula (2):

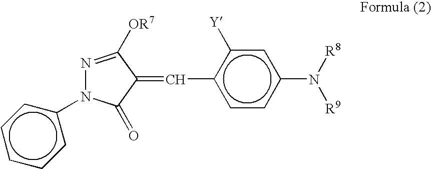

wherein Y′ represents a fluorine atom, a chlorine atom or a bromine atom; R⁷ represents a methyl group, an ethyl group, an n-propyl group or an isopropyl group; and R⁸ and R⁹ each independently represent a methyl group, an ethyl group, an n-propyl group, an isopropyl group or an allyl group.
(4) An ink sheet cartridge, which is loaded with the ink sheet as described in any one of the above items (1) to (3).
(5) A heat-sensitive transfer recording method, wherein the heat-sensitive transfer recording ink sheet as described in any one of the above items (1) to (3) is used with an image-receiving material having an ink-receiving layer containing a polymer on a support, to form an image.
(6) An arylidenepyrazolone dye compound represented by formula (2):

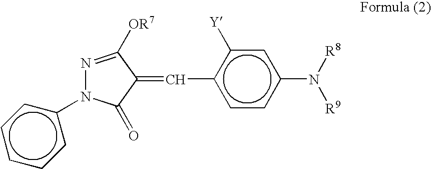

wherein Y′ represents a fluorine atom, a chlorine atom or a bromine atom; R⁷ represents a methyl group, an ethyl group, an n-propyl group or an isopropyl group; and R⁸ and R⁹ each independently represent a methyl group, an ethyl group, an n-propyl group, an isopropyl group or an allyl group.

The heat-sensitive transfer ink sheet of the present invention, the ink sheet cartridge of the present invention, the heat-sensitive transfer recording method of the present invention, and the arylidenepyrazolone dyes used therein of the present invention are described below in detail. The constitutional requirements described below may be embodied on the basis of the representative embodiments of the present invention. However the present invention is not limited to such embodiments.

In the present specification, “to” denotes a range including numerical values described before and after it as a minimum value and a maximum value.

### [Arylidenepyrazolone Dye Represented by Formula (1)]

To begin with, the arylidenepyrazolone dyes used in the present invention, which are represented by formula (1), are described in detail.

Arylidenepyrazolone dyes, which are similar to the arylidenepyrazolone dyes used in the present invention and represented by formula (1), are described in JP-A-2-3450. Specifically, the Compound Nos. 12 and 13 in JP-A-2-3450 are similar to the dye compound defined in the present invention. However, the document cited above makes no mention of the arylidenepyrazolone dyes having specific combinations of substituents, which are represented by formula (1), and it has never been known that the arylidenepyrazolone dyes of such specific structures are very useful for heat-sensitive transfer ink sheets in achieving the foregoing objects.

Arylidene Pyrazolone Dyes Described in JP-A-2-3450, which are Similar to the Dyes Used in the Present Invention

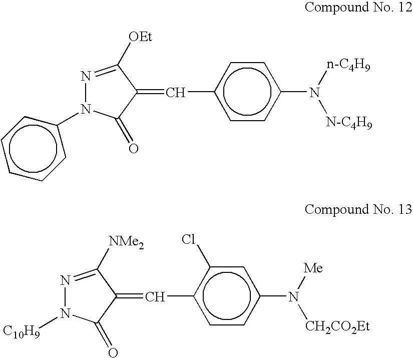

A structural feature of the arylidenepyrazolone dyes represented by formula (1) is to have an alkoxy group at the 5-position of the pyrazolone ring and a halogen atom at the ortho-position to the methine of the anilino group. The arylidenepyrazolone dyes having this feature of the present invention possess spectral characteristics desirable for color reproduction, high molecular extinction coefficients, high solubility and high fastness to light. Therefore, when they are used in heat-sensitive transfer recording ink sheets, the arylidenepyrazolone dyes of the present invention can deliver high transfer density, and also satisfy other performance requirements. So, the arylidenepyrazolone dyes of the present invention are especially suitable for use in heat-sensitive transfer recording.

In formula (1), X represents a substituted or unsubstituted alkoxy group having 1 to 6 carbon atoms. The substituent which the alkoxy group may have is described hereinafter.

In formula (1), Y represents a halogen atom.

In formula (1), R¹ represents a substituted or unsubstituted aryl group having 6 to 10 carbon atoms. Similarly to the above, the substituent which the aryl group may have is described hereinafter.

In formula (1), R², R⁵ and R⁶ each independently represent a hydrogen atom, an unsubstituted alkyl group having 1 to 4 carbon atoms, an unsubstituted alkoxy group having 1 to 4 carbon atoms, or a halogen atom.

In formula (1), R³ and R⁴ each independently represent a substituted or unsubstituted alkyl group having 1 to 6 carbon atoms, or an allyl group. Similarly to the above, the substituent which the alkyl group may have is described below.

The substituent that the groups represented by X, R¹, R³ and R⁴ may have is described below in more detail.

The substituent that the groups represented by X, R¹, R³ and R⁴ may have is not particularly limited. Examples thereof include a halogen atom, an alkyl group, an alkenyl group, an alkynyl group, an aryl group, a heterocyclic group, an alkoxy group, an aryloxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkyl- or aryl-sulfonylamino group, an alkylthio group, a sulfamoyl group, an alkyl- or aryl-sulfinyl group, an alkyl- or aryl-sulfonyl group, an acyl group, an aryloxycarbonyl group, an alkoxycarbonyl group, a carbamoyl group, an aryl- or heterocyclic-azo group, and an imido group.

The halogen atom that X, R¹, R³, and R⁴ may have includes a fluorine atom, a chlorine atom, a bromine atom, and an iodine atom. Of these, a chlorine atom and a bromine atom are preferable, a chlorine atom is particularly preferable.

The alkyl group that X, R¹, R³, and R⁴ may have includes a cycloalkyl group and a bicycloalkyl group. The alkyl group includes a substituted or unsubstituted, linear or branched alkyl group. The substituted or unsubstituted, linear or branched alkyl group is preferably an alkyl group having 1 to 30 carbon atoms. Examples of the alkyl group include a methyl group, an ethyl group, an n-propyl group, an isopropyl group, a t-butyl group, an n-octyl group, an eicosyl group, a 2-chloroethyl group, a 2-cyanoethyl group, or a 2-ethylhexyl group. The cycloalkyl group includes a substituted or unsubstituted cycloalkyl group. The substituted or unsubstituted cycloalkyl group is preferably a cycloalkyl group having 3 to 30 carbon atoms. Examples of the cycloalkyl group include a cyclohexyl group, a cyclopentyl group and a 4-n-dodecylcyclohexyl group. The bicycloalkyl group includes a substituted or unsubstituted bicycloalkyl group having 5 to 30 carbon atoms, i.e. a monovalent group obtained by removing one hydrogen atom from a bicycloalkane having 5 to 30 carbon atoms. Examples of the bicycloalkyl group include a bicyclo[1,2,2]heptan-2-yl group or a bicyclo[2,2,2]octan-3-yl group, and a tricyclo or higher structure having three or more ring structures. An “alkyl” group in a substituent described below (e.g. an “alkyl” group in an alkylthio group) represents such an “alkyl” group of the above concept.

The alkenyl group that X, R¹, R³, and R⁴ may have includes a cycloalkenyl group and a bicycloalkenyl group. The alkenyl group represents a substituted or unsubstituted, linear, branched, or cyclic alkenyl group. The alkenyl group is preferably a substituted or unsubstituted alkenyl group having 2 to 30 carbon atoms. Examples of the alkenyl group include a vinyl group, an allyl group, a prenyl group, a geranyl group, or an oleyl group. The cycloalkenyl group is preferably a substituted or unsubstituted cycloalkenyl group having 3 to 30 carbon atoms, i.e. a monovalent group obtained by removing one hydrogen atom from a cycloalkene having 3 to 30 carbon atoms. Examples of the cycloalkenyl group include a 2-cyclopenten-1-yl group or a 2-cyclohexen-1-yl group. The bicycloalkenyl group includes a substituted or unsubstituted bicycloalkenyl group, and preferably a substituted or unsubstituted bicycloalkenyl group having 5 to 30 carbon atoms, i.e. a monovalent group obtained by removing one hydrogen atom from a bicycloalkene having one double bond. Examples of the bicycloalkenyl group include a bicyclo[2,2,1]hept-2-en-1-yl group or a bicyclo[2,2,2]oct-2-en-4-yl group.

The alkynyl group that X, R¹, R³, and R⁴ may have is preferably a substituted or unsubstituted alkynyl group having 2 to 30 carbon atoms, e.g. an ethynyl group, or a propargyl group.

The aryl group that X, R¹, R³, and R⁴ may have is preferably a substituted or unsubstituted aryl group having 6 to 30 carbon atoms, e.g. a phenyl group, a p-tolyl group, a naphthyl group, an m-chlorophenyl group, or an o-hexadecanoylaminophenyl group.

The heterocyclic group that X, R¹, R³, and R⁴ may have is a monovalent group obtained by removing one hydrogen atom from a substituted or unsubstituted, aromatic or nonaromatic heterocyclic compound, which may be condensed to another ring. The heterocyclic group is preferably a 5- or 6-membered heterocyclic group. The hetero atom(s) constituting the heterocyclic group is preferably a nitrogen atom, a sulfur atom, or an oxygen atom. The heterocyclic group is more preferably a 5- or 6-membered aromatic heterocyclic group having 3 to 30 carbon atoms. The hetero ring in the heterocyclic group are exemplified below without denotation of their substitution sites: a pyridine ring, a pyrazine ring, a pyridazine ring, a pyrimidine ring, a triazine ring, a quinoline ring, an isoquinoline ring, a quinazoline ring, a cinnoline ring, a phthalazine ring, a quinoxaline ring, a pyrrole ring, an indole ring, a furan ring, a benzofuran ring, a thiophene ring, a benzothiophene ring, a pyrazole ring, an imidazole ring, a benzimidazole ring, a triazole ring, an oxazole ring, a benzoxazole ring, a thiazole ring, a benzothiazole ring, an isothiazole ring, a benzisothiazole ring, a thiadiazole ring, an isoxazole ring, a benzisoxazole ring, a pyrrolidine ring, a piperidine ring, a piperazine ring, an imidazolidine ring and a thiazoline ring.

The alkoxy group that X, R¹, R³, and R⁴ may have includes a substituted or unsubstituted alkoxy group. The substituted or unsubstituted alkoxy group is preferably an alkoxy group having 1 to 30 carbon atoms, e.g. a methoxy group, an ethoxy group, an isopropoxy group, an n-octyloxy group, a methoxyethoxy group, a hydroxyethoxy group, or a 3-carboxypropoxy group.

The aryloxy group that X, R¹, R³, and R⁴ may have is preferably a substituted or unsubstituted aryloxy group having 6 to 30 carbon atoms, e.g. a phenoxy group, a 2-methylphenoxy group, a 4-t-butylphenoxy group, a 3-nitrophenoxy group, or a 2-tetradecanoylaminophenoxy group.

The acyloxy group that X, R¹, R³, and R⁴ may have is preferably a formyloxy group, a substituted or unsubstituted alkylcarbonyloxy group having 2 to 30 carbon atoms, or a substituted or unsubstituted arylcarbonyloxy group having 7 to 30 carbon atoms, e.g. a formyloxy group, an acetyloxy group, a pivaloyloxy group, a stearoyloxy group, a benzoyloxy group, or a p-methoxyphenylcarbonyloxy group.

The carbamoyloxy group that X, R¹, R³, and R⁴ may have is preferably a substituted or unsubstituted carbamoyloxy group having 1 to 30 carbon atoms, e.g. an N,N-dimethylcarbamoyloxy group, an N,N-diethylcarbamoyloxy group, a morpholinocarbonyloxy group, an N,N-di-n-octylaminocarbonyloxy group, or an N-n-octylcarbamoyloxy group.

The alkoxycarbonyloxy group that X, R¹, R³, and R⁴ may have is preferably a substituted or unsubstituted alkoxycarbonyloxy group having 2 to 30 carbon atoms, e.g. a methoxycarbonyloxy group, an ethoxycarbonyloxy group, a t-butoxycarbonyloxy group, or an n-octylcarbonyloxy group.

The aryloxycarbonyloxy group that X, R¹, R³, and R⁴ may have is preferably a substituted or unsubstituted aryloxycarbonyloxy group having 7 to 30 carbon atoms, e.g. a phenoxycarbonyloxy group, a p-methoxyphenoxycarbonyloxy group, or a p-n-hexadecyloxyphenoxycarbonyloxy group.

The amino group that X, R¹, R³, and R⁴ may have includes an alkylamino group, an arylamino group, and a heterocyclic amino group. The amino group is preferably a substituted or unsubstituted alkylamino group having 1 to 30 carbon atoms, or a substituted or unsubstituted arylamino group having 6 to 30 carbon atoms, e.g. an amino group, a methylamino group, a dimethylamino group, an anilino group, an N-methyl-anilino group, a diphenylamino group, a hydroxyethylamino group, a carboxyethylamino group, a sulfoethylamino group, a 3,5-dicarboxyanilino group, or a 4-quinolylamino group.

The acylamino group that X, R¹, R³, and R⁴ may have is preferably a formylamino group, a substituted or unsubstituted alkylcarbonylamino group having 1 to 30 carbon atoms, or a substituted or unsubstituted arylcarbonylamino group having 7 to 30 carbon atoms, e.g. a formylamino group, an acetylamino group, a pivaloylamino group, a lauroylamino group, a benzoylamino group, or a 3,4,5-tri-n-octyloxyphenylcarbonylamino group.

The aminocarbonylamino group that X, R¹, R³, and R⁴ may have is preferably a substituted or unsubstituted aminocarbonylamino group having 1 to 30 carbon atoms, e.g. a carbamoylamino group, an N,N-dimethylaminocarbonylamino group, an N,N-diethylaminocarbonylamino group, or a morpholinocarbonylamino group. In the aminocarbonylamino group, the term “amino” has the same meaning as “amino” in the above-described amino group.

The alkoxycarbonylamino group that X, R¹, R³, and R⁴ may have is preferably a substituted or unsubstituted alkoxycarbonylamino group having 2 to 30 carbon atoms, e.g. a methoxycarbonylamino group, an ethoxycarbonylamino group, a t-butoxycarbonylamino group, an n-octadecyloxycarbonylamino group, or an N-methyl-methoxycarbonylamino group.

The aryloxycarbonylamino group that X, R¹, R³, and R⁴ may have is preferably a substituted or unsubstituted aryloxycarbonylamino group having 7 to 30 carbon atoms, e.g. a phenoxycarbonylamino group, a p-chlorophenoxycarbonylamino group, or an m-n-octyloxyphenoxycarbonylamino group.

The sulfamoylamino group that X, R¹, R³, and R⁴ may have is preferably a substituted or unsubstituted sulfamoylamino group having 0 to 30 carbon atoms, e.g. a sulfamoylamino group, an N,N-dimethylaminosulfonylamino group, or an N-n-octylaminosulfonylamino group.

The alkyl- or aryl-sulfonylamino group that X, R¹, R³, and R⁴ may have is preferably a substituted or unsubstituted alkylsulfonylamino group having 1 to 30 carbon atoms, or a substituted or unsubstituted arylsulfonylamino group having 6 to 30 carbon atoms, e.g. a methylsulfonylamino group, a butylsulfonylamino group, a phenylsulfonylamino group, a 2,3,5-trichlorophenylsulfonylamino group, or a p-methylphenylsulfonylamino group.

The alkylthio group that X, R¹, R³, and R⁴ may have is preferably a substituted or unsubstituted alkylthio group having 1 to 30 carbon atoms, e.g. a methylthio group, an ethylthio group, or an n-hexadecylthio group.

The sulfamoyl group that X, R¹, R³, and R⁴ may have is preferably a substituted or unsubstituted sulfamoyl group having 0 to 30 carbon atoms, e.g. an N-ethylsulfamoyl group, an N-(3-dodecyloxypropyl)sulfamoyl group, an N,N-dimethylsulfamoyl group, an N-acetylsulfamoyl group, an N-benzoylsulfamoly group, or an N-(N′-phenylcarbamoyl)sulfamoyl group.

The alkyl- or aryl-sulfinyl group that X, R¹, R³, and R⁴ may have is preferably a substituted or unsubstituted alkylsulfinyl group having 1 to 30 carbon atoms, or a substituted or unsubstituted arylsulfinyl group having 6 to 30 carbon atoms, e.g. a methylsulfinyl group, an ethylsulfinyl group, a phenylsulfinyl group, or a p-methylphenylsulfinyl group.

The alkyl- or aryl-sulfonyl group that X, R¹, R³, and R⁴ may have is preferably a substituted or unsubstituted alkylsulfonyl group having 1 to 30 carbon atoms, or a substituted or unsubstituted arylsulfonyl group having 6 to 30 carbon atoms, e.g. a methylsulfonyl group, an ethylsulfonyl group, a phenylsulfonyl group, or a p-toluenesulfonyl group.

The acyl group that X, R¹, R³, and R⁴ may have is preferably a formyl group, a substituted or unsubstituted alkylcarbonyl group having 2 to 30 carbon atoms, a substituted or unsubstituted arylcarbonyl group having 7 to 30 carbon atoms, or a substituted or unsubstituted heterocyclic carbonyl group having 4 to 30 carbon atoms and being bonded to said carbonyl group through a carbon atom, e.g. an acetyl group, a pivaloyl group, a 2-chloroacetyl group, a stearoyl group, a benzoyl group, a p-n-octyloxyphenylcarbonyl group, a 2-pyridylcarbonyl group, or a 2-furylcarbonyl group.

The aryloxycarbonyl group that X, R¹, R³, and R⁴ may have is preferably a substituted or unsubstituted aryloxycarbonyl group having 7 to 30 carbon atoms, e.g. a phenoxycarbonyl group, an o-chlorophenoxycarbonyl group, an m-nitrophenoxycarbonyl group, or a p-t-butylphenoxycarbonyl group.

The alkoxycarbonyl group that X, R¹, R³, and R⁴ may have is preferably a substituted or unsubstituted alkoxycarbonyl group having 2 to 30 carbon atoms, e.g. a methoxycarbonyl group, an ethoxycarbonyl group, a t-butoxycarbonyl group, or an n-octadecyloxycarbonyl group.

The carbamoyl group that X, R¹, R³, and R⁴ may have is preferably a substituted or unsubstituted carbamoyl group having 1 to 30 carbon atoms, e.g. a carbamoyl group, an N-methylcarbamoyl group, an N,N-dimethylcarbamoyl group, an N,N-di-n-octylcarbamoyl group, or an N-(methylsulfonyl)carbamoyl group.

Examples of the aryl- or heterocyclic-azo group that X, R¹, R³, and R⁴ may have include a phenylazo group, a 4-methoxyphenylazo group, a 4-pivaloylaminophenylazo group, and a 2-hydroxy-4-propanoylphenylazo group.

Examples of the imido group that X, R¹, R³, and R⁴ may have include an N-succinimido group and an N-phthalimido group.

X is preferably a substituted or unsubstituted alkoxy group having 1 to 5 carbon atoms, more preferably a substituted or unsubstituted alkoxy group having 1 to 3 carbon atoms, and most preferably an unsubstituted alkoxy group having 1 or 2 carbon atoms.

Y is preferably a fluorine atom, a chlorine atom or a bromine atom; more preferably a fluorine atom or a chlorine atom; and most preferably a chlorine atom.

R¹ is preferably a substituted or unsubstituted phenyl group, more preferably an unsubstituted phenyl group or a phenyl group substituted by an alkyl group, and most preferably an unsubstituted phenyl group.

R², R⁵ and R⁶ each are preferably a hydrogen atom, an unsubstituted alkyl group having 1 to 3 carbon atoms, an unsubstituted alkoxy group having 1 to 3 carbon atoms, or a chlorine atom; more preferably a hydrogen atom, an unsubstituted alkyl group having 1 or 2 carbon atoms, or an unsubstituted alkoxy group having 1 or 2 carbon atoms; and most preferably a hydrogen atom.

R³ and R⁴ each are preferably a substituted or unsubstituted alkyl group having 1 to 4 carbon atoms, or an allyl group; more preferably a substituted or unsubstituted alkyl group having 1 to 3 carbon atoms, or an allyl group, and most preferably an unsubstituted alkyl group having 2 to 3 carbon atoms, or an allyl group.

The following is an explanation about a preferable combination of various groups (atoms) in the dye represented by formula (1) may have: A preferred compound is a compound in which at least one of the groups is the above-described preferable group. A more preferred compound is a compound in which many various groups are the above-described preferable groups. The most preferred compound is a compound in which all groups are the above-described preferable groups.

More specifically, the preferable combination of the groups in formula (1) is a combination that X is an unsubstituted alkoxy group having 1 to 5 carbon atoms, Y is a fluorine atom, a chlorine atom or a bromine atom, R¹ is a substituted or unsubstituted phenyl group, R² is a hydrogen atom, R³ is a substituted or unsubstituted alkyl group having 1 to 4 carbon atoms, or an allyl group, R⁴ is a substituted or unsubstituted alkyl group having 1 to 4 carbon atoms, or an allyl group, R⁵ is a hydrogen atom and R⁶ is a hydrogen atom.

The more preferable combination is a combination that X is an unsubstituted alkoxy group having 1 to 4 carbon atoms, Y is a fluorine atom, a chlorine atom or a bromine atom, R¹ is a substituted or unsubstituted phenyl group, R² is a hydrogen atom, R³ is a substituted or unsubstituted alkyl group having 1 to 3 carbon atoms, or an allyl group, R⁴ is a substituted or unsubstituted alkyl group having 1 to 3 carbon atoms, or an allyl group, R⁵ is a hydrogen atom and R⁶ is a hydrogen atom.

The most preferable combination is a combination that X is an unsubstituted alkoxy group having 1 to 3 carbon atoms, Y is a fluorine atom, a chlorine atom or a bromine atom, R¹ is a substituted or unsubstituted phenyl group, R² is a hydrogen atom, R³ is an unsubstituted alkyl group having 1 to 3 carbon atoms, or an allyl group, R⁴ is an unsubstituted alkyl group having 1 to 3 carbon atoms, or an allyl group, R⁵ is a hydrogen atom and R⁶ is a hydrogen atom, or a combination as shown in formula (2) which represents an arylidenepyrazolone dye.

### [Arylidenepyrazolone Dye Represented by Formula (2)]

The arylidenepyrazolone dyes represented by formula (2) are described below in detail.

In addition to the structural feature of the arylidenepyrazolone dyes represented by formula (1), the arylidenepyrazolone dyes represented by formula (2) further have a feature that their molecular weight is 410 or less from the viewpoint of thermal diffusion, and dyes combining these features have never been known. Among the arylidenepyrazolone dyes represented by formula (1), the arylidenepyrazolone dyes represented by formula (2) having these features are superior in transfer sensitivity in particular, so they are especially suitable for usage in the heat-sensitive transfer recording ink sheet.

Y′ in formula (2) is a fluorine atom, a chlorine atom or a bromine atom. Y′ is preferably a fluorine atom or a chlorine atom, and more preferably a chlorine atom.

R⁷ in formula (2) is a methyl group, an ethyl group, an n-propyl group or an isopropyl group. R⁷ is preferably a methyl group, an ethyl group or an n-propyl group, more preferably a methyl group or an ethyl group, and most preferably an ethyl group.

R⁸ and R⁹ in formula (2) each independently represent a methyl group, an ethyl group, an n-propyl group, an isopropyl group or an allyl group. Each of them is preferably an ethyl group, an n-propyl group, an isopropyl group or an allyl group, more preferably an ethyl group, an n-propyl group or an allyl group, and most preferably an ethyl group or an allyl group.

The following is an explanation about a preferable combination of various groups (atoms) in the dye represented by formula (2) may have: A preferred compound is a compound in which at least one of the groups is the above-described preferable group. A more preferred compound is a compound in which many various groups are the above-described preferable groups. The most preferred compound is a compound in which all groups are the above-described preferable groups.

The preferable combination of the groups in formula (2) is a combination that Y′ is a fluorine atom or a chlorine atom, R⁷ is a methyl group or an ethyl group, R⁸ is an ethyl group, an n-propyl group or an allyl group, and R⁹ is an ethyl group, an n-propyl group or an allyl group.

The more preferable combination is a combination that Y′ is a chlorine atom, R⁷ is a methyl group or an ethyl group, R⁸ is an ethyl group, an n-propyl group or an allyl group, and R⁹ is an ethyl group, an n-propyl group or an allyl group.

The most preferable combination is a combination that Y′ is a chlorine atom, R⁷ is an ethyl group, R⁸ is an ethyl group or an allyl group, and R⁹ is an ethyl group or an allyl group.

Specific examples of the arylidenepyrazolone dye represented by formula (1) according to the present invention are illustrated below, but these examples should not be construed as limiting the arylidenepyrazolone dyes of formula (1) which can be used in the present invention. Incidentally, Me, Et, n-Pr, Ph, n-Bu and i-Pr in the following examples stand for a methyl group (—CH₃), an ethyl group (—CH₂—CH₃), an n-propyl group (—CH₂—CH₂—CH₃), a phenyl group (—C₆H₅), an n-butyl group (—CH₂—CH₂—CH₂—CH₃) and an isopropyl group (—CH(CH₃)₂), respectively.

TABLE 1
Dye represented by formula (1)

| No | X | Y | R¹ | R² | R³ | R⁴ | R⁵ | R⁶ |
|---|---|---|---|---|---|---|---|---|
| 1 | OEt | Cl | Ph | H | Et | Et | H | H |
| 2 | OEt | F | Ph | H | Et | Et | H | H |
| 3 | OEt | Br | Ph | H | Et | Et | H | H |
| 4 | OEt | Cl | Ph | H | n-Pr | n-Pr | H | H |
| 5 | OEt | Cl | Ph | H | Allyl | Allyl | H | H |
| 6 | OMe | Cl | Ph | H | Et | Et | H | H |
| 7 | OMe | Cl | Ph | H | n-Pr | n-Pr | H | H |
| 8 | OMe | Cl | Ph | H | Me | i-Pr | H | H |
| 9 | O(n-Pr) | Cl | Ph | H | Et | Et | H | H |
| 10 | O(n-Pr) | Cl | Ph | H | n-Pr | n-Pr | H | H |
| 11 | O(n-Pr) | Cl | Ph | H | n-Bu | n-Bu | H | H |
| 12 | OEt | Cl | Ph | H | n-Bu | n-Bu | H | H |

The exemplified compound Nos. 1 to 10 in the above table are included in concrete examples of the arylidenepyrazolone dye represented by formula (2).

These arylidenepyrazolone dyes can be synthesized by generally-used dehydrating condensation reaction of a pyrazolone derivative and an aminobenzaldehyde derivative.

More specifically, the arylidenepyrazolone dyes can be easily synthesized, e.g., by heating a pyrazolone derivative represented by formula (3) and an aminobenzaldehyde derivative represented by formula (4) in a solvent, such as methanol, in accordance with the following reaction scheme 1 and making these derivatives undergo dehydrating condensation reaction. Details of these syntheses are illustrated in Examples.

The pyrazolone derivative represented by formula (3) can be easily synthesized, e.g., by the method described in J. Heterocycl. Chem., 1996, 33(2), p. 479.

The aminobenzaldehyde derivative represented by formula (4) can be easily synthesized, e.g., by the method described in Jikken Kagaku Koza (Courses in Experimental Chemistry), 4th Ed., vol. 20, pp. 284-288, or the method described in Jikken Kagaku Koza (Courses in Experimental Chemistry), 4th Ed., vol. 21, pp. 113-118.

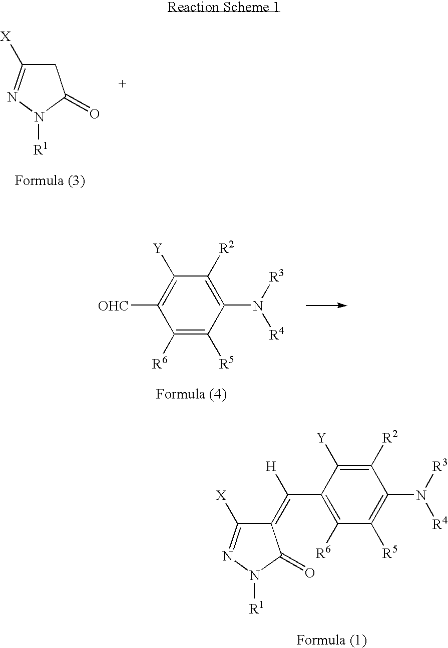

X and R¹ in formula (3) have the same meanings as X and R¹ in formula (1), respectively. Y and R² to R⁶ in formula (4) have the same meanings as Y and R² to R⁶ in formula (1), respectively. Formula (1) in the reaction scheme 1 is the same as the foregoing formula (1).

### [Heat-Sensitive Transfer Recording Ink Sheet]

It is preferable that the arylidenepyrazolone dyes described above are used as yellow of three primary colors. The maximum absorption wavelength of the arylidenepyrazolone dye defined in the present invention is preferably 400 to 480 nm, and more preferably 420 to 460 nm.

The heat-sensitive transfer recording ink sheet of the present invention is characterized in that the above-described arylidenepyrazolone dye represented by formula (1) is contained in the ink sheet. The heat-sensitive transfer recording ink sheet generally has a structure composed of a support and a dye-providing layer formed on the support. The arylidenepyrazolone dye represented by formula (1) is contained in the dye-providing layer. The heat-sensitive transfer recording ink sheet of the present invention can be manufactured by the steps of preparing an ink solution by dissolving the arylidenepyrazolone dye represented by formula (1) in a solvent with a binder or by dispersing the arylidenepyrazolone dye as fine particles, and forming a dye-providing layer by coating the ink solution on the support, followed by drying, if necessary.

As the support (base material sheet) that can be used for the heat-sensitive transfer recording ink sheet of the present invention, use can be made of any support arbitrarily selected from ordinary supports that have been used for an ink sheet. For example, there can be preferably used such materials as described in paragraph No. 0050 of JP-A-7-137466. A thickness of the support is preferably in the range of from 2 μm to 30 μm.

As a binder resin that can be used for the dye-providing layer in the heat-sensitive transfer recording ink sheet of the present invention, there is no particular limitation of its kind, in so far as the binder resin has such excellent heat resistance that the binder resin does not hinder transfer of dyes to an image-receiving layer. Examples of preferable binders resins include such materials as described in paragraph No. 0049 of JP-A-7-137466. Similarly, a solvent for forming the dye-providing layer can be arbitrarily selected for use from ordinary solvents. Specifically, such a solvent as described in Example of JP-A-7-137466 can be preferably used.

A content of the arylidenepyrazolone dye represented by formula (1) in the dye-providing layer is preferably in the range of from 0.03 g/m² to 1.0 g/m², more preferably from 0.1 g/m² to 0.6 g/m². A thickness of the dye-providing layer is preferably in the range of from 0.2 μm to 5 μm, more preferably from 0.4 μm to 2 μm.

The heat-sensitive transfer recording ink sheet of the present invention may have a layer(s) other than the dye-providing layer. For example, there may be an interlayer between the support and the dye-providing layer, or there may be a back layer on the surface of the support (hereinafter, such the surface is sometimes referred to as a “back surface”) opposite to the dye-providing layer. Examples of the interlayer include a subbing layer and a diffusion-preventing layer to prevent the diffusion of dyes in the dye-providing layer through the support (a hydrophilic barrier layer). An example of the back layer is a heat resistant slip layer, and by which a thermal head can be prevented from adhesion to the ink sheet.

In order to use the above heat-sensitive transfer recording ink sheet of the present invention as a heat-sensitive recording material capable of recording a full-color image, it is preferred that a cyan ink sheet containing a thermally diffusible cyan dye which can form a cyan image, a magenta ink sheet containing a thermally diffusible magenta dye which can form a magenta image, and a yellow ink sheet containing a thermally diffusible yellow dye which can form a yellow image be formed on a support by applying these sequentially. In addition to the above ink sheets, an ink sheet containing a black-image-forming substance may be further formed as required.

As a cyan ink sheet containing a thermally diffusible cyan dye capable of forming a cyan image, there can be preferably used such ink sheets as described in, for example, JP-A-3-103477 and JP-A-3-150194.

As a magenta ink sheet containing a thermally diffusible magenta dye capable of forming a magenta image, there can be preferably used such ink sheets as described in, for example, Japanese patent Nos. 2542921 and 2747874.

The ink sheet of the present invention that is used as a heat-sensitive transfer recording material can be loaded with an ink sheet cartridge. As to the structure and loading method of the ink sheet cartridge, those which can be ordinary adopted in the field of heat-sensitive transfer recording can also be used in the present invention. More specifically, the ink sheet cartridge arts described, e.g., in JU-A-63-161851 (“JU-A” means unexamined published Japanese utility model application), JU-A-63-161851 and JU-A-1-101864 can be applied to the present invention. Of these arts, the art described in JU-A-1-101864 is especially preferred.

### (Heat-Sensitive Transfer Recording)

When a heat-sensitive transfer recording is performed using the heat-sensitive transfer recording ink sheet of the present invention, a heating tool such as a thermal head and an image-receiving sheet are used in combination with the heat-sensitive transfer recording ink sheet. Specifically, the image recording is achieved according to a process in which a thermal energy transferred from a thermal head in accordance with image recording signals is given to an ink sheet, and then a dye in the portion to which the thermal energy was given is transferred to an image-receiving sheet and fixed therein. As a composition and a usable material of the image-receiving sheet, such compositions and materials as described in paragraph Nos. 0056 to 0074 of JP-A-7-137466 can be preferably used.

According to the present invention, it is possible to provide a heat-sensitive transfer recording ink sheet and a heat-sensitive transfer recording method satisfying excellent color reproduction, high image storability and high transfer density in print samples by use of a specific arylidenepyrazolone dye. Further, according to the present invention, it is possible to provide novel specific arylidenepyrazolone dyes having especially high molecular extinction coefficient, fastness to light and high solubility in a solvent, among the aforementioned arylidenepyrazolone dyes.

The present invention can provide a novel arylidenepyrazolone dye having spectral characteristics desirable for color reproduction, high molecular extinction coefficient, high solubility, high transfer density and high fastness to light. In addition, the present invention can provide heat-sensitive transfer recording ink sheets containing such an arylidenepyrazolone dye and a heat-sensitive transfer recording method utilizing such a dye. Moreover, the present invention can provide a heat-sensitive transfer recording ink sheet and a heat-sensitive transfer recording method satisfying excellent color reproduction, high image storability and heat-sensitive transfer in high density.

The present invention will be described in more detail based on the following examples. The materials, the amounts to be used, the proportions, the details and procedures of treatment or processing, which will be shown in the following working examples, may be appropriately changed or modified, without departing from the spirit of the present invention. Therefore, the following examples are not interpreted as limiting of the scope of the present invention.

### EXAMPLES

### Example 1

### Synthesis of Arylidenepyrazolone Dye

### <Synthesis of Exemplified Compound (1)>

The exemplified compound (1) was synthesized according to the following scheme 1.

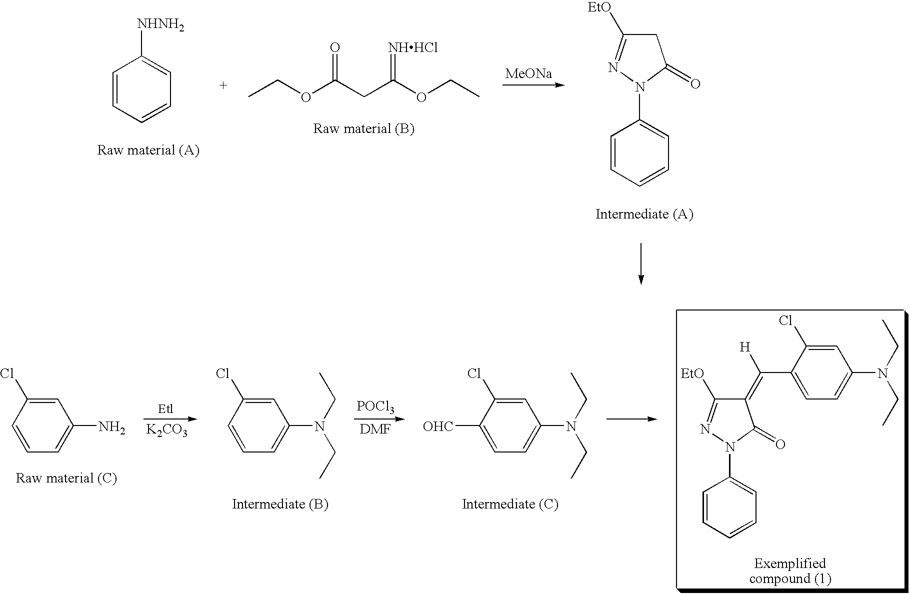

### Synthesis of Intermediate (A)

While being cooled in an ice bath, 50 ml of ethanol was stirred at an internal temperature of 10° C. or below, and thereto 19.6 g of the raw material (B) was added, and then 10.8 g of the raw material (A) was added dropwise. The resultant mixture was further stirred for one hour as it was cooled in the ice bath, and thereto 58 g of a 28% methanol solution of sodium methoxide (SM-28) was added dropwise while keeping the temperature at 10° C. or below. After stirring was continued for additional two hours, 54 ml of 6N hydrochloric acid was added dropwise to the reaction mixture at a temperature of 20° C. or below, and further stirred for 30 minutes. Crystals thus precipitated were separated by suction filtration, washed with water, and then dried. Thus, 14.2 g of white crystals (Intermediate(A)) were obtained (yield: 70%).

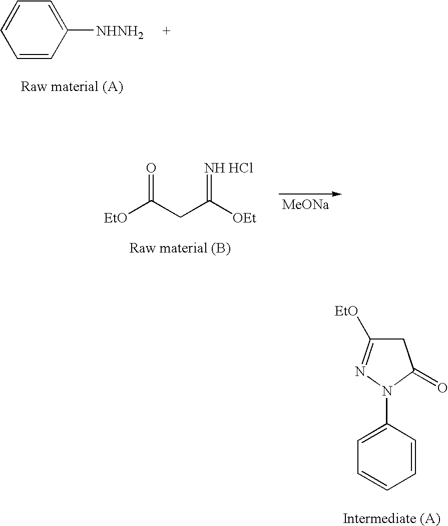

### Synthesis of Intermediate (B)

In a stream of nitrogen gas, 63.8 g of 3-chloroaniline, 173 g of potassium carbonate and 100 mL of 1-methyl-2-pyrrolidone were stirred at 65° C., and thereto 120 mL of iodoethane was added dropwise. The resultant mixture was heated under reflux for 4 hours, and then cooled to room temperature. Thereto 300 mL of ethyl acetate was added. And the mixture was washed with 600 mL of a 5% saline solution, and further washed with a 10% saline solution. Then, the resultant ethyl acetate layer was separated from the aqueous layer again, then dried over magnesium sulfate, and further concentrated by evaporating the solvent under a reduced pressure. Thus, 92 g of Intermediate (B) was obtained.

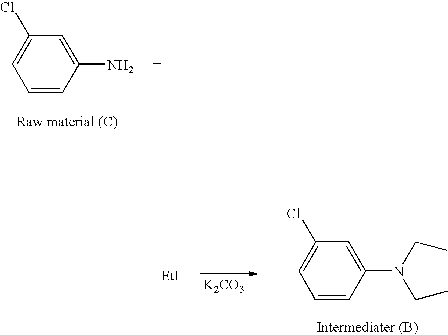

### Synthesis of Intermediate (C)

Toluene in an amount of 140 mL was added to 46.4 mL of DMF (N,N-dimethyformamide), and they were stirred while cooling in an ice-methanol bath. Thereto, 41.3 mL of phosphorus oxychloride was added dropwise at a temperature of 10° C. or below. Thereafter, the mixture was stirred for 30 minutes at temperatures ranging from 15° C. to 20° C., and thereto 55.1 g of Intermediate (B) was added dropwise at a temperature of from 15° C. to 20° C. Then, the resultant mixture was heated up to 100° C. and subjected to reaction for 2 hours. After the reaction solution was cooled down to 60° C., 50 mL of acetonitrile was added, and the mixture was cooled in an ice-methanol bath. Furthermore, 400 mL of water was added dropwise at a temperature of 20° C. or below, and then 320 g of a 25% aqueous solution of sodium hydroxide was added dropwise at a temperature of 20° C. or below. Thereto, 400 mL of water and 750 mL of ethyl acetate were further added, and extraction was carried out. The resultant organic layer was separated, washed with a saline solution, then dried over anhydrous magnesium sulfate, and further concentrated by means of a rotary evaporator. Thus, 61.4 g of Intermediate (C) was obtained (yield: 96.7%).

The Intermediate (C) obtained was used in the next step without purification.

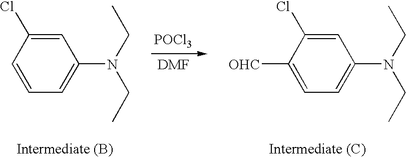

### Synthesis of Exemplified Compound (1)

A mixture of 14.2 g of Intermediate (A), 19.6 g of Intermediate (C), 5.4 g of ammonium acetate and 90 mL of isopropyl alcohol was stirred and heated under reflux for 1 hour. After cooling to 50° C., the mixture was subjected to filtration for removal of dust. Further, the resultant was cooled to room temperature, and stirred for 1 hour. Crystals thus precipitated were separated by suction filtration under a reduced pressure, and washed by rising them with a small amount of isopropyl alcohol, thereby giving 21.2 g of the exemplified compound (1) (yield: 94.6%).

### <Syntheses of Exemplified Compounds (2), (3) and (5)>

The exemplified compounds (2), (3) and (5) were synthesized referring to the method adopted in the foregoing synthesis example.

Maximum absorption wavelengths of the exemplified compounds (1), (2), (3) and (5) obtained herein, which were each measured in a state of ethyl acetate solution (concentration: 1×10⁻⁶ mol/L, light path length: 10 mm), and melting points and chemical shift values of ¹H NMR spectra (300 MHz) of these exemplified compounds are shown in the following Table 2.

In addition, other exemplified compounds, namely the exemplified compounds (4) and (6) to (12), were also synthesized referring to the method in the foregoing synthesis examples.

TABLE 2

| | | Maximum | |
|---|---|---|---|
| | Melting | absorption | |
| | point | wavelength (Ethyl | |
| | (° C.) | acetate) (nm) | ¹H NMR(ppm) |
| Exemplified | 130 | 436.3 | δ (DMSO-d₆) 9.6-8.5 (m, 1H), |
| compound (1) | | | 8.0 (s, 1H), 7.9 (d, 2H), 7.4 (t, 2H), |
| | | | 7.15 (t, 1H), 6.9 (d, 1H), 6.8 (d, 1H), |
| | | | 4.45 (q, 2H), 3.5 (q, 4H), 1.45 (t, 3H), |
| | | | 1.15 (t, 6H) |
| Exemplified | 178 | 430.2 | δ (DMSO-d₆) 8.5 (t, 1H), 7.9 (d, 2H), |
| compound (2) | | | 7.75 (s, 1H), 7.4 (t, 2H), 7.1 (t, 1H), |
| | | | 6.7 (d, 1H), 6.65 (q, 1H), 4.45 (q, 2H), |
| | | | 3.5 (q, 4H), 1.5 (t, 3H), 1.15 (t, 6H) |
| Exemplified | 153 | 436.5 | δ (DMSO-d₆) 8.5 (d, 1H), 7.95 (s, 1H), |
| compound (3) | | | 7.9 (d, 2H), 7.4 (t, 2H), 7.15 (t, 1H), |
| | | | 7.05 (s, 1H), 6.85 (d, 1H), 4.45 (q, 2H), |
| | | | 3.5 (q, 4H), 1.45 (t, 3H), 1.15 (t, 6H) |
| Exemplified | 102 | 428.4 | δ (DMSO-d₆) 8.45 (d, 1H), 8.0 (s, 1H), |
| compound (5) | | | 7.9 (d, 2H), 7.4 (t, 2H), 7.15 (t, 1H), |
| | | | 6.9 (s, 1H), 6.8 (d, 1H), |
| | | | 5.8-6.0 (m, 2H), 5.1-5.3 (m, 4H), |
| | | | 4.45 (q, 2H), 4.1 (d, 4H), 1.45 (t, 3H) |

### Example 2

### Production and Evaluation of Heat-Sensitive Transfer Recording Ink Sheet

### <Production of Heat-Sensitive Transfer Recording Ink Sheet>

A polyester film of 6.0 μm in thickness (trade name: Lumirror, manufactured by Toray Industries, Inc.), a back surface of which had been subjected to a heat resistant lubricating treatment with a 1 μm thick thermosetting acrylic resin, was used as a support. On the front surface of the film, the following dye-providing layer-coating composition was coated by a wire bar coating so that a dry thickness of the dye-providing layer became 1 μm. Thus, Ink sheet 1 was prepared.

(Dye-providing layer-coating composition)

| | |
|---|---|
| Exemplified compound (1) | 5.0 parts by mass |
| Polyvinylbutyral resin | 5.0 parts by mass |
| (Trade name: ESLEC BH-6, manufactured by Sekisui | |
| Chemical Co., Ltd.) | |
| Methyl ethyl ketone/toluene (1/1, at mass ratio) | 90 parts by mass |

Ink sheets 2, 3 and ink sheets for comparison 4 to 7 were produced in the same manner as in the production of the ink sheet 1, except that the above exemplified compound (1) was altered to the following dye as shown in Table 3, respectively.

<Image recording and Evaluation>

The thus-obtained ink sheet 1 and an image-receiving sheet ASK2000 (trade name, manufactured by FUJIFILM Corporation) were superposed so that the dye-providing layer in the ink sheet and the image-receiving layer in the image-receiving sheet were contacted with each other. For printing, a thermal head was used from the back side of the dye-providing material under the following conditions: Output of the thermal head: 0.25 W/dot; Pulse interval: 0.15 milliseconds to 15 milliseconds; and dot density: 6 dots/mm.

Then, the image-receiving layer of the image-receiving sheet was image-wise colored by the magenta dye. Thereby a brilliant image recording was achieved with no unevenness of transfer printing. Image recordings were conducted in the same manner as the above-described image recording, except that ink sheet 1 was replaced by ink sheets 2 to 7, respectively.

First, each of the thus-image-recorded heat-sensitive transfer image-receiving materials was subjected to irradiation with Xenon light (17000 lux) for 3 days, to examine light stability (fastness to light) of the dye image. Status A reflection densities after irradiation were measured in the portions indicating Status A reflection density of 1.0, and a stability of the image was evaluated in terms of a residual ratio (percentage) based on the reflection density (1.0) before irradiation, according to the following three grades:
A: 95% or more
B: 80% or more, but less than 95%
C: less than 80%

With respect to the transferability, status A reflection densities were measured in the portion indicating a solid density (100% dot density) of each of the obtained images, and evaluated according to the following three grades:
A (very good): reflection density of 2.2 or more
B (good): reflection density of 1.8 or more but less than 2.2
C (probably acceptable): reflection density of 1.8 or more but less than 1.6

Storage stability of the ink sheets obtained in the foregoing manner was evaluated as follows. Each ink sheet was wound into a roll, and allowed to stand for 24 hours under conditions that the temperature and the humidity were kept at 60° C. and 70% RH, respectively. Prints were made using each ink sheet before and after this forced aging, respectively, and examined for changes in reflection density by visual observations. And these changes were evaluated according to the following three grades.
A (very good): Almost no change in reflection density
B (acceptable): A little change in reflection density
C (unacceptable): A great change in reflection density

The thus-obtained results are shown in Table 3.

TABLE 3

| | | | | Storage | |
|---|---|---|---|---|---|
| | | | | stability | |
| Ink sheet | | Light | Transfer- | of ink | |
| No. | Dye | stability | ability | sheet | Remarks |
| 1 | (1) | A | A | A | This |
| | | | | | invention |
| 2 | (2) | A | A | A | This |
| | | | | | invention |
| 3 | (5) | A | A | A | This |
| | | | | | invention |
| 4 | Dye for | A | C | B | Comparative |
| | comparison 1 | | | | example |
| 5 | Dye for | C | A | B | Comparative |
| | comparison 2 | | | | example |
| 6 | Dye for | A | B | B | Comparative |
| | comparison 3 | | | | example |
| 7 | Dye for | A | B | B | Comparative |
| | comparison 4 | | | | example |

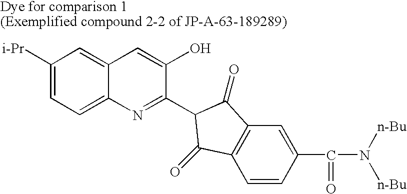

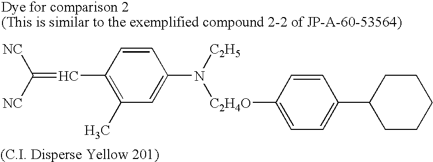

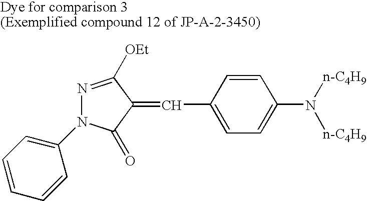

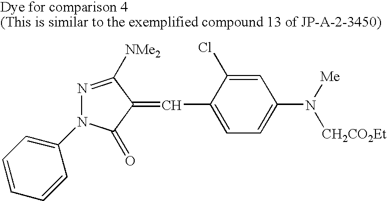

Herein, the Dye for comparison 4 is a compound comparable to the Exemplified Compound 13 of JP-A-2-3450, wherein a phenyl group is used in place of a group —C₁₀H₉ as the substituent attached to the nitrogen atom of the pyrazolone ring because the group —C₁₀H₉ cannot be identified.

In addition, those dyes were each examined for molecular extinction coefficient (L mol⁻¹cm⁻¹) in an ethyl acetate solution, and it was found that the molecular extinction coefficient of the exemplified compound (1) was 52,800, that of the exemplified compound (2) was 61,900, that of the exemplified compound (5) was 53,700, that of the dye for comparison 1 was 55,100 and that of the dye for comparison 2 was 52,900.

The results of the foregoing image-recording tests demonstrate that the arylidenepyrazolone dyes represented by formula (1) defined in the present invention have high molecular extinction coefficient and are superior to the known similar dyes in every performance including light stability, transferability and ink-sheet storage stability.

The test results further demonstrate that the images transferred to image-receiving layers from the ink sheets using the dyes defined in the present invention in particular provide high transfer densities as compared with the case of using the dye for comparison 1 and have high fastness to light as compared with the case of using the dye for comparison 2.

On the other hand, of the samples having undergone 24-hour aging under conditions that the temperature was kept at 60° C. and the humidity at 70% RH for the purpose of evaluating ink-sheet storage stability, while the ink sheets using the dye for comparison 1, 3 or 4 were clearly distinguished by crystallization of the dye, the ink sheets using the dye defined in the present invention were free of crystallized dyes.

It can be seen from these results that the arylidenepyrazolone dye defined in the present invention can satisfy various performance capabilities required for an ink sheet because they give high coefficients of molecular extinction, high fastness to light, high transfer density and high storage stability of an ink sheet compared with the comparative dyes.

Having described our invention as related to the present embodiments, it is our intention that the invention not be limited by any of the details of the description, unless otherwise specified, but rather be construed broadly within its spirit and scope as set out in the accompanying claims.

What we claim is:

1. A heat-sensitive transfer recording ink sheet, comprising a base material sheet and a dye-providing layer formed on one side of the base material sheet, wherein at least one kind of yellow dye contained in the dye-providing layer is an arylidenepyrazolone dye represented by formula (1): 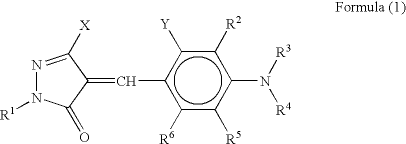
wherein X represents a substituted or unsubstituted alkoxy group having 1 to 6 carbon atoms; Y represents a halogen atom; R¹ represents a substituted or unsubstituted aryl group having 6 to 10 carbon atoms; R², R⁵ and R⁶ each independently represent a hydrogen atom, an unsubstituted alkyl group having 1 to 4 carbon atoms, an unsubstituted alkoxy group having 1 to 4 carbon atoms, or a halogen atom; R³ and R⁴ each independently represent a substituted or unsubstituted alkyl group having 1 to 5 carbon atoms, or an allyl group; and R³ and R⁴ and/or R⁵ and R⁶ may combine with each other to form a 5- or 6-membered ring.

2. The heat-sensitive transfer recording ink sheet according to claim 1, wherein X is an unsubstituted alkoxy group having 1 to 3 carbon atoms, Y is a fluorine atom, a chlorine atom or a bromine atom, R¹ is a substituted or unsubstituted phenyl group, R², R⁵ and R⁶ each are a hydrogen atom, and R³ and R⁴ each are a substituted or unsubstituted alkyl group having 1 to 3 carbon atoms, or an allyl group.

3. The heat-sensitive transfer recording ink sheet according to claim 1, wherein the arylidenepyrazolone dye represented by formula (1) is an arylidenepyrazolone dye represented by formula (2): 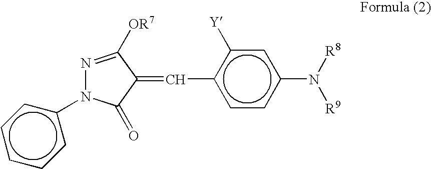
wherein, Y′ represents a fluorine atom, a chlorine atom or a bromine atom; R⁷ represents a methyl group, an ethyl group, an n-propyl group or an isopropyl group; and R⁸ and R⁹ each independently represent a methyl group, an ethyl group, an n-propyl group, an isopropyl group or an allyl group.

4. An ink sheet cartridge, which is loaded with the ink sheet according to claim 1.

5. A heat-sensitive transfer recording method, wherein the heat-sensitive transfer recording ink sheet according to claim 1 is used with an image-receiving material having an ink-receiving layer containing a polymer on a support, to form an image.

6. An arylidenepyrazolone dye compound represented by formula (2): 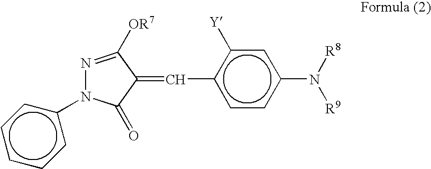
wherein, Y′ represents a fluorine atom, a chlorine atom or a bromine atom; R⁷ represents a methyl group, an ethyl group, an n-propyl group or an isopropyl group; and R⁸ and R⁹ each independently represent a methyl group, an ethyl group, an n-propyl group, an isopropyl group or an allyl group.
